(19) United States
(12) Patent Application Publication
(10) Pub. No.: US 2009/0221096 A1
(43) Pub. Date: Sep. 3, 2009

(54) THIN FILM BIOSENSOR AND METHOD AND DEVICE FOR DETECTION OF ANALYTES

(75) Inventor: Anthony R. Torres, Centerville, UT (US)

Correspondence Address: BRINKS HOFER GILSON & LIONE/UTAH;UTAH OFFICE, 405 South Main Street, Suite 800, SALT LAKE CITY, UT, 84111-3400, US

(73) Assignee: Great Basin Scientific, Salt Lake City, UT (US)

(21) Appl. No.: 12/096,817

(22) PCT Filed: Dec. 13, 2006
(86) PCT No.: PCT/US06/62034

§ 371 Date: Oct. 14, 2008

Related U.S. Application Data
(60) Provisional application No. 60/749,871, filed on Dec. 13, 2005.
(60) Provisional application No. 60/749,976, filed on Dec. 13, 2005.
(60) Provisional application No. 60/788,315, filed on Mar. 31, 2006.
(60) Provisional application No. 60/788,314, filed on Mar. 31, 2006.

(51) Int. Cl. G01N 33/53; G01N 21/00; B05D 1/36; G01N 33/543

(52) U.S. Cl. 436/501; 422/57; 427/261; 436/518

(57) ABSTRACT

Thin-film biosensor chips for detecting a target analyte in a biological sample are disclosed. The chips include a solid substrate, an antireflective optical layer, an attachment layer using a non-polymeric silane, and an Fc-specific binding molecule coupled to the non-polymeric silane. Kits containing the chips and methods of using and making the chips are also disclosed.

### CROSS REFERENCE TO RELATED APPLICATIONS

This application claims the benefit of the filing date of International Application Number PCT/US2006/062034, filed Dec. 13, 2006, U.S. Provisional Patent Application No. 60/749,871, filed Dec. 13, 2005, U.S. Provisional Patent Application No. 60/749,976, filed Dec. 13, 2005, U.S. Provisional Patent Application No. 60/788,314, filed Mar. 31, 2006, and U.S. Provisional Patent Application No. 60/788,315, filed Mar. 31, 2006, the disclosures of which are incorporated, in their entirety, by this reference.

### TECHNICAL FIELD OF THE INVENTION

The present invention relates to devices and methods for detecting target analytes in a biological sample based upon the alteration of light characteristics associated with binding of the analyte to a thin-film biosensor chip.

### BACKGROUND OF THE INVENTION

The use of thin-film biosensor chips for point-of-care diagnostic applications is well-known in the art. Thin-film biosensor chips produce a detectable attenuation of the spectral characteristic of light impinging on the chip by “thin-film” phenomenon. The thin film phenomenon is used to detect the presence or absence of an analyte of interest by detecting a change in color associated with an increase in thickness associated with the binding of the analyte of interest to the chip. The amount of analyte of interest that binds to the chip can also be determined by quantitation of film thickness.

U.S. Pat. No. 5,955,377 (Maul et al.) discloses methods and kits for detection of an analyte of interest in a sample using a thin-film based assay. The thin-film biosensor chips described by Maul et al. generally include a light reflective or transmissive substrate supporting one or more layers forming a thin film, the thin film comprising an attachment layer and a receptive layer which specifically binds the analyte of interest.

While such devices have been found useful as a rapid point-of-care diagnostic assay for various infectious diseases, there is a need in the art to improve the sensitivity of such chips, so as to detect target analytes that are present in biological samples in low abundance.

### SUMMARY OF THE INVENTION

The present invention relates generally to thin-film biosensor chips, methods of using thin-film biosensor chips to conduct thin-film biological assay methods for detecting the presence or absence of a target analyte in a biological sample, kits containing such thin film biosensor chips, and methods of preparing thin-film biosensor chips.

In one aspect, the present invention relates to a thin-film biosensor chip for detecting a target analyte in a biological sample, comprising a solid substrate, an antireflective optical layer coating the substrate, and an attachment layer comprising a non-polymeric silane non-covalently coupled to the antireflective optical layer. and an Fc-specific binding molecule coupled to the non-polymeric silane.

In some embodiments, the thin-film biosensor chip includes one or more optional components. One optional component is an amino-functional polypeptide layer coupled to the attachment layer. The amino-functional polypeptide layer may have a repeating phenylalanine-lysine subunit (also called poly(phenylalanine-lysine). In some embodiments, the thin-film biosensor chip includes an Fc-specific binding molecule. In some embodiments, the Fc-specific binding molecule is selected from the group consisting of protein G, protein A, protein L, protein LA, C1q complement protein, Fc receptor protein, IgG3 binding protein M12, anti-Fc antibodies, and recombinant proteins that specifically bind Fc. In some embodiments, the Fc-specific binding molecule is protein G. In some embodiments, the Fc-specific binding molecule is coupled to the attachment layer. In some embodiments, the Fc-specific binding molecule is coupled to the polypeptide layer.

In some embodiments, the thin-film biosensor chips further include an analyte binding layer coupled to the attachment layer. In some embodiments, the analyte binding layer may be coupled to the polypeptide layer. In some embodiments, the analyte binding layer may be coupled to the Fc-binding molecule. The analyte binding layer comprises one or more analyte-specific binding molecules. In some embodiments, the analyte binding layer comprises a first binding molecule, wherein the first binding molecule can bind a target analyte. In some embodiments, the analyte binding layer comprises a second binding molecule that can bind a second target analyte. In some embodiments, the second binding molecule can bind the same target analyte which binds to the first binding molecule. In some embodiments, the analyte binding layer comprises a plurality of binding molecules that can bind a plurality of target analytes. In some embodiments, the first binding molecule is coupled to the attachment layer. In some embodiments, the first binding molecule is coupled to the polypeptide layer. In some embodiments, the first binding molecule is coupled to the Fc-specific binding molecule.

In some embodiments, the first binding molecule is non-covalently coupled to the attachment layer. In some embodiments, the first binding molecule is covalently coupled to the attachment layer. In some embodiments, the first binding molecule is non-covalently coupled to the polypeptide layer. In some embodiments, the first binding molecule is covalently coupled to the polypeptide layer. In some embodiments, the first binding molecule is non-covalently coupled to the Fc-specific binding molecule. In some embodiments, the first binding molecule is covalently coupled to the Fc-specific binding molecule.

In some embodiments, the first binding molecule is a protein. In some embodiments, the first binding molecule is an antibody. In some embodiments, the first binding molecule is a polyclonal antibody and the second binding molecule is a monoclonal antibody.

In some embodiments, the thin-film biosensor chip also comprises a reflective layer coating the substrate and underlying the antireflective optical layer. The reflective layer may be a material with a refractive index of between about 3.8 and about 4.0. In some embodiments, the reflective layer comprises amorphous silicon.

In some embodiments, the substrate comprises a material selected from the group consisting of aluminum, alumina, silicon, silica, glass, and polycarbonate. In some embodiments, the antireflective layer may be a material selected from silicon nitride and diamond-like carbon. In some embodiments the antireflective layer is silicon nitride.

In some embodiments, the non-polymeric silane contains an amine group. In some embodiments, the non-polymeric silane is selected from the group consisting of aminoalkyltrialkoxysilane and amidoalkyltrialkoxysilane. In some embodiments, the non-polymeric silane is a 3-aminopropyltrialkoxysilane. In some embodiments, the non-polymeric silane is 3-aminopropyltriethoxysilane.

In another aspect, the present invention relates to a kit for a thin-film biosensor assay for detecting a target analyte in a biological sample, comprising a thin film biosensor chip, as described above. The kit containing a thin-film biosensor chip may also have a first analyte-specific binding molecule capable of binding to the chip. In some embodiments, the kit further comprises a reagent which when mixed with the target analyte bound to the biosensor chip precipitates on the biosensor chip resulting in a detectable change in mass.

In another aspect, the present invention relates to a method of preparing a thin-film biosensor chip for detecting a target analyte in a biological sample, comprising providing a solid substrate, coating the substrate with an antireflective optical layer, contacting the antireflective optical layer with a non-polymeric silane. In some embodiments, the non-polymeric silane is suspended in a solvent when contacted with the antireflective layer. In some embodiments, the method includes removing the solvent.

The thin-film biosensor prepared in the method may have a number of optional components, as already briefly described above. In some embodiments, the method optionally includes contacting the non-polymeric silane with a first analyte-specific binding molecule capable of binding the target analyte. In some embodiments, the method optionally includes contacting the non-polymeric silane with a second analyte-specific binding molecule capable of binding a second target analyte. In some embodiments, the method optionally includes coating the substrate with a reflective layer underlying the antireflective optical layer.

In some embodiments, the method of preparing the thin-film biosensor further comprises adding a reagent which when mixed with the target specific analyte bound to the biosensor chip precipitates on the biosensor chip resulting in detectable change in mass.

In another aspect, the present invention also relates to a thin-film biological assay method for detecting the presence or absence of a target analyte in a biological sample, comprising (a) providing a thin film biosensor chip, as described above, (b) contacting the chip with a biological sample. In some embodiments, the method of detecting the target analyte also includes (c) evaluating a change in mass associated with the target analyte binding to the analyte-specific binding molecule. In other embodiments, the method also includes the step of mixing the biological sample with a blocking agent prior to contacting the chip with a biological sample, and then combining the mixture with an analyte-specific binding molecule.

In some embodiments, the method of detecting a target analyte includes providing a first analyte-specific binding molecule capable of binding the target analyte. In some embodiments, the method of detecting a target analyte includes providing a second analyte-specific binding molecule capable of binding a second target analyte. In some embodiments, the method includes contacting the chip with a reagent which when mixed with the target-specific analyte bound to the biosensor chip results in a detectable change in mass. In some embodiments, the method includes exposing the chip to light. In some embodiments, the light is polarized.

### BRIEF DESCRIPTION OF THE DRAWINGS

FIGS. 1A (with unreacted biosensor surface) and 1B (with reacted biosensor surface) are diagrams showing the interference phenomena associated with the deposition of a mass on a biosensor surface.

FIGS. 2A and 2B are diagrams showing specular (FIG. 2A) and non-specular or diffuse (FIG. 2B) surfaces.

FIGS. 3A-F are diagrams showing cross-sectional representations of various biosensor surfaces. FIGS. 3A-C show instrumentally read surfaces. FIGS. 3D-F show visually read surfaces. Materials and layers are designated as follows: substrate (1), optical thin film (2), attachment layer (3), receptive material (4), reflective layer (5), metal film (6), and composite interference film (7).

### DETAILED DESCRIPTION OF THE INVENTION

### Definitions

While the terminology used in this application is standard within the art, the following definitions of certain terms are provided to assure clarity.

Units, prefixes, and symbols may be denoted in their SI accepted form. Unless otherwise indicated, nucleic acids are written left to right in 5′ to 3′ orientation. Numeric ranges recited herein are inclusive of the numbers defining the range and include and are supportive of each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the case of any amino acid or nucleic sequence discrepancy within the application, the figures control.

The term “Fc-specific binding molecule” means a molecule that is capable of specifically binding to the Fc region of an immunoglobulin molecule.

The term “polymer” means a chain of molecules consisting of structural units and repeating units connected by a covalent chemical bond.

The term “silane” means a chemical compound containing a silicon atom without a polymeric chain of repeating subunits.

Examples of non-polymeric silanes include but are not limited to organosilanes, aminosilanes, vinylsilanes, epoxysilanes, methacrylsilanes, sulfursilanes, alkylsilanes, polyalkylsilanes, (alkyl)alkoxysilanes, aminoalkylsilanes, (aminoalkyl)alkoxysilanes (such as (3-aminopropyl)triethoxysilane), and the like.

The term “siloxane” means a chemical compound containing a silicon-oxygen-silicon (Si—O—Si) molecular unit.

The term “inorganic reactivity” when used in reference to compounds with a silicon atom, means the ability to form covalent bonds between oxygen and silicon atoms resulting in a siloxane-type molecular unit (Si—O—Si).

The term “organic reactivity” when used in reference to compounds with a silicon atom, means the ability to form bonds or interactions with another chemical entity not directly involving silicon and oxygen atoms or a siloxane-type molecular unit (Si—O—Si).

The term “diamond-like carbon” also abbreviated “DLC” means amorphous carbon materials that display some of the properties of natural diamond and contain significant amounts of sp³ hybridized carbon atoms.

As used herein, the term “target-specific binding molecule,” “analyte-specific binding molecule,” “first binding molecule,” and “second binding molecule” may be used interchangeably and refer to a molecule capable of binding a target analyte.

Applications

A number of optical thin film monitoring technologies include ellipsometry, multiple angle reflectometry, interference spectroscopy, profilometry, surface plasmon resonance, evanescent wave, and various other forms or combinations of polarimetry, reflectometry, spectroscopy, and spectrophotometry. With these monitoring technologies, detecting or measuring changes in thickness, density, or mass of thin films can be obtained in an assay involving concentration-dependent immobilization of one or more analytes on surface suitably selected with binding material. These thin film assay technologies can directly detect or quantitate the analyte of interest, and are alternatives to conventional solid phase assays.

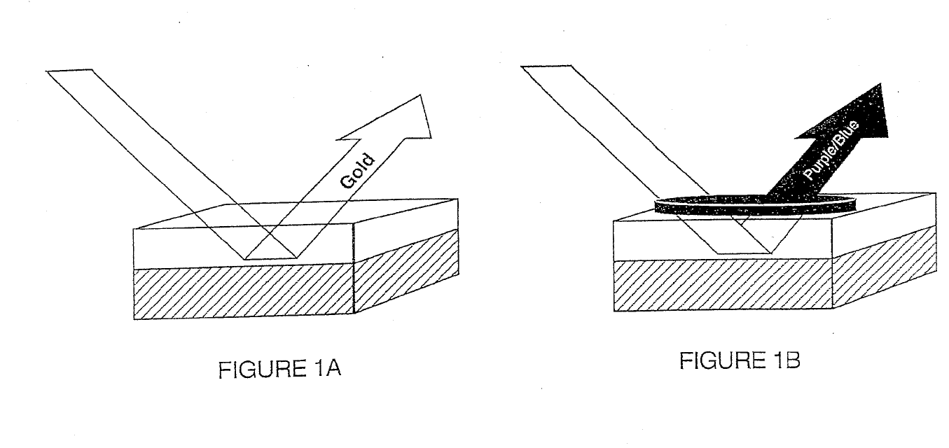

FIGS. 1A and 1B show the general phenomenon of light interference that is an aspect of the utility of thin film monitoring. This phenomenon is generally independent of the macroscopic surface characteristics of the biosensor device. For example, the phenomenon can cause a change in the observed color of light reflected from the surface without providing any specific pattern on the surface, such as a diffraction grating or other pattern. Generally the surface is a planar surface with no specific pattern. In another aspect, the surface may be provided in a shape or design that can be visually useful to the human eye. An unreacted biosensor surface causes white light incident at the device to be reflected as gold light, whereas a reacted biosensor surface, due to the additional matter (or mass) from analyte binding will cause the incident white light to be reflected as purple, blue, or some other color of light. The change from gold to purple or blue indicates the interference difference between the reacted and the unreacted biosensor surfaces.

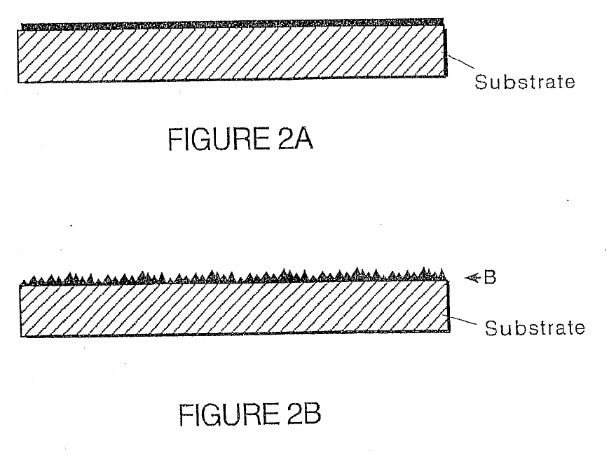

FIGS. 2A and 2B show the specular biosensor surface (2A) and a non-specular or diffuse biosensor surface (2B). The more specular the biosensor surface, the greater the probability that an analyte will bind to the chip and the more homogeneous the interference pattern will appear. Thus, the more uniform the biosensor surface, the more sensitive an optical assay may be performed.

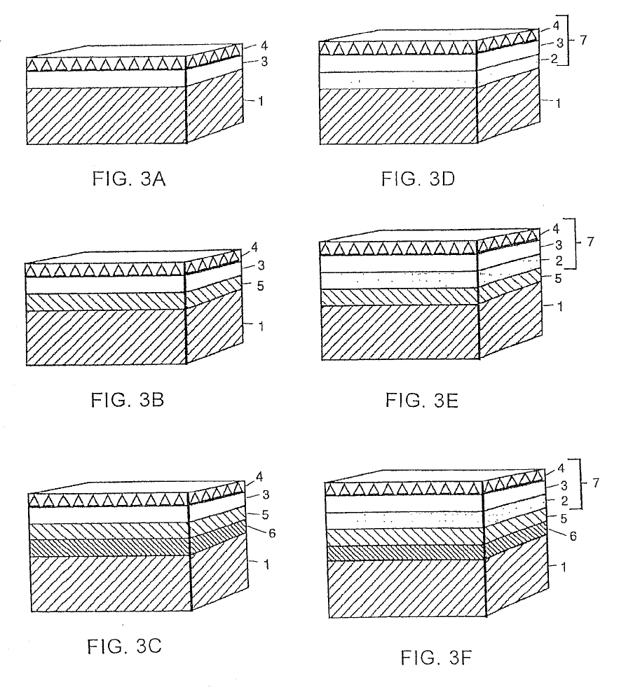

FIGS. 3A-F show the general structure of various types of biosensor surfaces that can be utilized. For an instrument-read device the surface is provided with a substrate, an attachment layer, and a receptive material layer, and may also be provided with amorphous silicon and/or a metal film. In contrast, for visually readable devices it is necessary to provide an optical thin film (or an interference film) which, together with the attachment layer and binding layer (receptive material layer), form a composite interference film. These various layers and their interactions are discussed in more detail below.

Substrate

One or more thin films on a surface may attenuate incident light on that surface producing a change in the incident light that may be measured either by reflectance or transmittance. Reflection occurs when light encounters a medium of a different refractive index than the ambient medium. In many applications, the ambient medium is air with a refractive index of 1.0. Transmission is a general term describing the process by which incident light leaves a surface or medium on a side other than the incident surface. The transmittance of a medium is the ratio of the transmitted light to that of the incident light. Both the reflected or transmitted light can be detected visually or may be measured with an instrument. The actual structure of a chosen device depends on whether a reflection or transmission mode is desired, and whether the result is to be interpreted visually or instrumentally. These specific combinations can be relevant to the choice of a substrate(s) and are described generally below.

Visually Observed Reflectance

One aspect of the thin film phenomenon may be understood as the interference colors observed when viewing oil on water on an asphalt surface. This phenomenon can also be seen in a piece of multilayered mica, a fragment of ice, a stretched plastic bag, or a soap film. An observed change in color can be due to local variations in the thickness of the material. The variety of visual colors observed when an oil layer is on water is due to the difference in refractive index between water and oil. The color observed is further intensified because the water (the underlying layer) provides a mirror-like (specular) reflection. When the water and oil are on an asphalt surface, the asphalt absorbs transmitted light, suppressing back reflection, which would tend to dilute the colors observed. The eye is more sensitive to contrast than to changes in intensity; therefore, material selection can augment or amplify the production of high contrast colors as a result of mass or thickness change at the surface. Films may be added to the surface of a material to modify the reflectance of one or more wavelengths or band of wavelengths. These types of materials are often used to produce sunglasses, camera lenses, and solar windowpanes.

When the biosensor surface is designed to produce a visual color change, the optical substrate can provide a surface that is reflective only at its uppermost surface, and of a known refractive index. Polished, monocrystalline silicon, metals including but not limited to Ag on quartz, TiN/Si02/Si, TiN, gallium arsenide on germanium, and Zi sulfice on silicon, and some ceramics or dark glasses, glass, glass with a rough backside, CaF2, plastics, and BK7 glass can provide surfaces which may be used directly as a substrate. In some embodiments, the substrate serves as both a substrate and a reflective layer. In other embodiments, the substrate is not a reflective layer. Materials that contribute to the generation of an observed signal may be considered optically active.

Use of some materials such as glass or plastics may require additional processing before use as a substrate. For example, glass will allow reflection to occur at its upper and back surfaces. To avoid such dual reflection, and to enable use of such materials, an additional film can be applied to the uppermost surface. Amorphous silicon, a thin metal film, or a combination of these materials may be used. In this case, the glass serves as a solid support and is not involved in the generation of a reflected light or observed color. In this situation, the substrate can be considered optically passive.

The refractive index at the uppermost surface influences what optical thin film or antireflective coating to apply or use when either a single substrate material or a more complex structure is used (see below). With a monocrystalline silicon substrate the uppermost surface of the substrate is considered. With a substrate comprising transparent glass coated with amorphous silicon, the amorphous silicon surface is considered. When using a reflective substrate to produce a color change perceived by the eye, the addition of a film of suitable refractive index can assist when determining which wavelengths of light are anti-reflected (absorbed).

The optical substrate materials may produce a specular reflection, or may be treated to, or intrinsically produce, a diffuse reflection which is less angle dependent in viewing the signal, as discussed more fully below.

Visually Observed Transmission

For this technique, color observed in an assay is not viewed as reflected light, but is observed as the light is transmitted through a surface. Materials for selective transmission of light have been used to produce sunglasses, camera lenses, windowpanes, and narrowbandpass filters. The materials can selectively reflect and transmit different wavelengths of light. For example, a narrowbandpass filter will reflect a large band of wavelengths of light, and will selectively transmit only a very small band of wavelengths centered around one specific wavelength. The narrowbandpass filter is constructed of an optical glass which is coated on one side with a material which will reflect many wavelengths of light. A change in the thickness of the material which coats the optical glass will change the useful range of the filter centering on a new set of wavelengths.

In one aspect, the optical substrate selected can be transmissive to the visible wavelengths of light. Materials such as monocrystalline silicon, metals, certain plastics, and ceramics are not suitable unless they are extremely thin, transparent sections. Glasses and certain transparent plastics are the most useful for this application. In this type of technique the substrate is optically active. For the generation of a color change visible to the eye, the refractive index of the substrate impacts the type of antireflective film which is selected. A uniform or smooth surface assists to prevent loss of signal due to scattering at one or more of the transmitting surfaces.

A glass substrate coated with a layer of amorphous silicon may be transmissive to visible light at certain angles, if the amorphous silicon layer is sufficiently thin. This is also true for a very thin layer of metal on a glass substrate. For this type of biosensor surface, the viewing should be arranged such that the amorphous silicon is the back surface of the biosensor piece (i.e., opposite to the viewing surface).

Instrumentally Observed Reflection

The use of an antireflective or optical thin-film component can be optional when making observations with an instrument. A reflectometer detects color change or change in luminosity (intensity) for generating a signal. This color change may be different from the color change selected visual detection, as the instrument will record changes in intensity and does not require a maximal change in contrast. Antireflective film thickness may be adjusted to provide for the maximal change in recorded intensity as a function of analyte binding. In addition, modifying a reflectometer can allow measured changes in color/luminosity (intensity) with a specularly reflecting or diffusing reflecting surface.

In ellipsometric measurements, the optical substrate can provide a specular reflection. Reflection should occur only from the uppermost surface. As previously discussed, glass can serve as a support substrate in this case and is optically passive. Instrumental detection can observe a change in light intensity due to changes in reflected light from thin film surfaces. The light may be elliptically or linearly polarized, polychromatic, monochromatic, and of any wavelength desired.

Instrumentally Observed Transmission

An optical substrate which is transparent to the incident light may be used, whether that light is polychromatic, monochromatic, linearly polarized, or elliptically polarized, and of any wavelength desired. Use of an antireflective film is optional, but if required for use with a reflectometer, the guidelines discussed in visual detection can apply as well. Thus, the refractive index of the optical substrate influences the selection of the antireflective coating. The design of the reflectometer can be easily modified to allow reflection or transmission measurements to be made.

When a change in the transmitted light is to be made independent of any color, an antireflective film can be omitted. Oftentimes, the substrate may permit transmission of some component or components of the incident light. A change in mass or character on the uppermost surface of the biosensor piece can affect the transmitted light in a detectable manner. Materials such as the Irtran series produced by Eastman Kodak may be of use in this application for monitoring changes in the infrared (IR) properties of these films.

Thus, the term “substrate” includes not only a solid surface for holding the layers described hereafter, but also an optically active substrate which may included as a component in an optical thin film. For clarity, these two portions of a substrate are discussed separately, but those in the art will recognize that the layers (to which the attachment layer and other layers are attached) may be optically active to provide a detectable change when there is a change in thickness or mass of the thin film. The substrate can be a solid material and can support a layer of material which acts optically. The optically active material can have a known refractive index if it is to be combined with an optical thin film to produce an interference effect. Thus, the optically active material may be formed from any desired material which is reflective or made reflective, as discussed below. For instrument use, the substrate can also be transparent (e.g., glass or plastic) so that transmitted light is analyzed.

In one aspect, the optical substrate can be formed of, or have coated on it, a material that provides either diffuse or specular reflection. The substrate may be rigid or flexible, reflective or transmissive. The substrate may form an optically functional component of the biosensor surface. The substrate may act as an optically passive support (and be provided with optically active layers). Devices designed for instrumental analysis may omit an antireflective (optical thin film) coating on the substrate, while those designed for viewing by eye may include such a coating. Criteria useful for selecting an optical substrate for instrument applications, or for visual color-signal generating application, are presented below.

A wide range of support imparting materials may form the optical substrate, including glass, fused silica, plastic, ceramic, metal, and semiconductor materials. The substrate may be of any thickness desired. Flexible optical substrates include thin sheets of plastic and like materials. Most substrates can be modified using solvent, plasma etching, or acid cleaning before subsequent layers are deposited.

For color-signal generation visible to the eye, an antireflective coating material may be used. Polymer films, such as mylar (polyethylene teraphthalate) and other materials having a low surface energy may not adhere well to substrate materials prompting additional substrate treatment before deposition of an antireflective layer. To improve adhesion, optical substrates may be etched in an oxygen plasma, under conditions standard for oxygen plasma cleaning in semiconductor processing.

The surfaces of many solid materials, such as glass, and semiconductor materials, such as silicon, metals, etc., can be sufficiently smooth to provide specular reflection. In some embodiments, those surfaces can be further polished. Reflection-based assays can occur with reflection at an upper surface. Visual detection often will often be assisted with an anti-reflection layer which can be added or otherwise deposited on a substrate by vapor deposition of a thin metal film on the substrate, and attachment of subsequent layers by techniques appropriate for those layers. For example, the uppermost surface of a glass substrate may be coated with a layer to prevent unwanted reflections from the lower surface.

Metal Layer

If the substrate is to be used in a reflection mode, and is partially or fully transparent, it may be coated with an opaque material to block transmitted light and allow reflection to occur only from the upper surface. For example, a glass substrate may be coated with a layer of aluminum, chromium, or other transparent conducting oxide, by mounting in a vacuum chamber facing an aluminum-filled tungsten boat. The chamber is evacuated to a pressure of 1×10-5 Torr. Current is passed through the tungsten boat, raising it to a temperature at which the aluminum deposits on the substrate at a rate of 20 Å/second for 100 seconds, coating the glass with an opaque layer of aluminum having a thickness of 2000 Å. Thinner layers of aluminum or chromium may also be used to eliminate any back surface reflections. Non-conducting deposition techniques may be used to deposit the metal film.

Amorphous Silicon

The aluminum-coated glass, described above, may be considered optically passive. Thus, if it is coated with a layer of hydrogenated amorphous silicon, the optical characteristics of the substrate will be derived from a substance such as amorphous silicon. The aluminum-coated glass can be used when the amorphous silicon deposition process includes a conducting surface. Techniques which involve the use of a non-conducting surface for the deposition of amorphous silicon are also known. To produce this substrate, the aluminum-coated glass can be mounted on one of two opposing electrodes in plasma-enhanced chemical vapor deposition system. The system is evacuated, and the substrates are heated to 250° C. A constant flow of silane (SiH4) gas into the chamber raises the pressure to 0.5 Torr. A plasma is struck by applying 10 mW/cm2 of RF power to the electrodes. A film of amorphous silicon deposits on the substrates, and grows to a thickness of approximately 1000 nm in about 75 minutes. The amorphous silicon so formed may form the first optically functional layer on the biosensor surface.

A glass substrate coated only with amorphous silicon (without the aluminum layer) may also be useful. Transparent substrates, such as glass, fused silica, sapphire, and many plastics may be used in instrument transmission measurements, without additional modification. Visual color-signal generation is possible with a transmissive substrate where the anti-reflection properties of the coatings are determined from the transmitted light.

Many of the substrates with a sufficiently reflective surface for thin-film measurements are metals. Examples of these metals, include but are not limited to, iron, stainless steel, nickel, cobalt, zinc, gold, copper, aluminum, silver, titanium, etc. and alloys thereof. Metal substrates can be used when an instrumental method is employed. For instrumental systems, the substrate can be reflective and planar. In contrast, visible color signal generation can be very difficult, but not impossible, because of the challenge in matching the reflectivity of the metal with a suitable antireflective coating. The reflectivity of the optical substrate and the optical thin film (see below) used can match for the optimal production of an interference color. Thus, devices designed for color production can include amorphous silicon-coated metal substrates as discussed above.

The surface topography, and hence fuzziness or irregularity may be characterized with a surface profilometer, such as the Dek-tak® (Sloan Technology Corp., Santa Barbara, Calif.). The Dek-tak® provides readings on the separation or distance between surface features and an average value for the height of surface features over a defined region of a surface. One useful measure of the surface is the Root Mean Square (RMS) or average surface roughness divided by the average peak spacing, where a peak is defined to be a protrusion with a height of at least 50% of the RMS roughness. Since roughness is a function of the reflectivity versus angle, it may be quantified by measuring the angle dependence of the reflectivity. For a light source incident at 30° from normal, the reflected light intensity on a photodiode should be measured as a function of the angle from 0° to 90°. The wafer selected should optimally show a smoothly varying reflectivity over the angular range viewed.

The substrate material may be cut, sawed, scribed, laser scribed, or otherwise manipulated into the desired biosensor piece configuration. Suitable biosensor pieces for a single use assay can be of any desired size, for example from 0.5 cm2 to 1 cm2. Biosensor piece sizes are not restricted to the above, as alternative formats may require substantially more or less reactive biosensor surface.

Optional Optical Thin Film Material(s)

FIGS. 1A and 1B show the simplest form of a single optical thin film, having a substrate coated with a thin layer of material such that reflections from the outer surface of the film and the outer surface of the substrate cancel each other by destructive interference. Two requirements exist for exact cancellation of reflected light waves. First the reflections can be 180° out of phase and, second, they can be of equal amplitude or intensity.

In the reflection mode, the optical thin-film properties of the coatings can suppress the reflection of some wavelengths of light and enhance the reflection of others. This causes the suppressed wavelengths of incident light to enter the substrate, or an opaque coating on the substrate where they are absorbed. Most of the light of other wavelengths, whose reflection is not suppressed, do not enter the coated substrate and is reflected; however, some components may be absorbed. As the optical thickness of the coating changes, the range of wavelengths in the reflected light changes. In transmission mode, the properties of the coatings suppress the reflection of some wavelengths of light and enhance the reflection of others, as in the reflection mode. This causes the suppressed wavelengths of the incident light to enter the substrate and to be transmitted. Light of other wavelengths, whose reflection is not suppressed to as great an extent, is reflected and transmitted to a lesser extent. As the optical thickness of the coating changes, the range of wavelengths in the transmitted light changes.

Where eye-visible color-signal generation is desired (see FIGS. 3D-F), the assay result may also be measured by instrumentation. For the production of an interference film with an optical substrate, the substrate should have a refractive index of the square of the refractive index of the receptor layer, i.e., (1.5)² or 2.25. The material selected can be mechanically stable to subsequent processes, reflective, and of known refractive index. It is not always possible to match the optical substrate to a particular film, for example, a biological film. In these cases, an intermediate optical thin film can be used to compensate for the lack of a suitable optical substrate. For eye-visible color-signal generation, the substrate material can adhere to the optical thin film material, and second, in the simplest case, the refractive index of the substrate can approximately equal the square of the refractive index of the material directly above it. For example, use of a silicon wafer with a refractive index of approximately 4.1 allows a biosensor surface to be designed with a wide variety of corresponding optical thin films or antireflective materials. The material can be coated to a thickness of a quarterwave for the wavelengths to be attenuated. Other substrate materials can be used as a biosensor surface when they both adhere and possess an appropriate refractive index.

The optical thin-film coating can be deposited onto the surface of the substrate by many coating techniques, for example, by sputtering or by vapor phase deposition in a vacuum chamber. Various other useful coating techniques are known to those skilled in the art. Materials useful as optical thin-film coatings can be formed of clear material which is significantly transmissive at the thickness utilized, and suppresses some wavelength of reflective light when coated onto the substrate. The film, once deposited onto the optical substrate, can also be stable to subsequent processes.

For example, a substrate such as a polished silicon wafer has a refractive index of approximately 4.1. The optical thin film material selected can have an index of refraction of approximately 2.0 (i.e., close to the square root of 4.1). Maximal “apparent” color change is achieved for silicon with materials having refractive indices near 2.0, such as silicon nitride (Si3N4) or silicon/silicon dioxide composites. Other optical thin film materials that have a similar refractive index include, but are not limited to: tin oxide, zinc oxide, chromium oxide, barium titanate, cadmium sulfide, manganese oxide, lead sulfide, zinc sulfide, zirconium oxide, nickel oxide, aluminum oxide, boron nitride, magnesium fluoride, iron oxide, silicon oxynitride (SixOyNz) (also known as native oxides), boron oxide, lithium fluoride, titanium oxide, calcium fluoride, SiON, silver on quartz, TiN/Si02/Si, TiN, gallium arsenide on germanium, Zi sulfide on silicon, poly Si, Sib2, Si substrate, silicon carbide and the like.

Silicon Nitride

One method for the deposition of silicon nitride is a plasma-enhanced chemical vapor deposition technique similar to that described above for the deposition of amorphous silicon. This technique (and modifications of this technique) is suitable for the deposition of a large number of materials. For example, to produce Si3N4, ammonia (NH3) gas is added to silane gas. Silicon nitride performs well as an optical thin film on substrates of monocrystalline silicon and polycrystalline silicon, or on amorphous silicon and polycrystalline silicon with optically passive substrates.

The compatibility of the silicon nitride deposition process with the amorphous silicon deposition process can result a very cost-effective combination. The two films may be deposited as follows. Glass substrates are mounted in an evaporation system where a 2000 Åthick layer of aluminum is deposited on the glass, as described above. Then the substrates are mounted in a plasma-enhanced chemical vapor deposition system, where a 1 micron thick layer of amorphous silicon is deposited, as described above, followed by a silicon nitride layer. In this way an inexpensive reflection-mode biosensor surface is formed on a glass substrate. This approach may be extended to the deposition of these coatings on dielectrics and flexible substrates described in U.S. Pat. No. 3,068,510, issued Dec. 18, 1962, to Coleman incorporated herein by reference in its entirety.

The refractive index of the silicon nitride, or by analogy the silicon/silicon dioxide composites, may be controlled in the vapor deposition process. The ratio of gases may be varied, or the deposition rates may be varied, and a variety of other methods known to those skilled in the art may be used to control or select the refractive index of the optical thin film deposited.

Multi-Layer Films

Multi-layer optical thin-film coatings may be deposited by electron beam evaporation. A substrate is mounted in a vacuum deposition chamber and suspended over two or more crucibles of the various materials to be evaporated. Each crucible is then heated by an electron-beam gun, and the rate of evaporation monitored using a crystal thickness monitor. Each crucible is covered by a movable shutter. By alternately opening and closing the shutters, the substrate is exposed sequentially to each vapor stream, until the desired multi-layer stack has been deposited, or a multi-component film is deposited. The described procedure may be generalized to more than two crucibles in order to deposit multiple layers of various optical thin film materials, or multi-component films tailored to a specific refractive index.

The biosensor surface when coated at a specific thickness with a silicon nitride film suppresses certain wavelengths in the blue range of visible light and therefore reflects a yellow-gold interference color. Although a yellow-gold interference color is utilized in some examples, the interference color of the biosensor surface can be any suitable color in the spectrum of light. The color depends on the substrate material selected, the chemical composition and refractive index of the optical layer/s selected, and the thickness and number of coated layers. These design techniques can also be utilized to produce biosensor surfaces with signals or backgrounds in the ultraviolet (UV) or infrared region of the spectrum of light, however, these biosensor surfaces are useful only in instrumented detection of a bound analyte since UV and infrared light is not visually detected.

For example, lithium fluoride may form one component of a multi-layer stack. It has a refractive index of 1.39 for visible light, and thus forms a one-quarter wavelength layer for green light at a thickness of 925 Å. It may be evaporated from a platinum crucible at approximately 900° C.

Titanium Film

Titanium films can be useful for the production of optical films. Such films have advantages since they use materials which are safer to handle and dispose of than other optical materials, such as SiH4. The method of application can also be more cost effective and rapid with less instrumentation required.

Titanium dioxide has a refractive index of approximately 2.2 for visible light, and thus forms a one-quarter wavelength layer for green light at a thickness of 585 Å. Because titanium dioxide decomposes into lower oxides upon heating, the evaporated films are not stoichiometric. To deposit stoichiometric titanium dioxide, the electron-beam can be pulsed. The deposition occurs at approximately 2000° C.

Organotitanates may be hydrolyzed to titanium dioxide, (TiO2) under conditions which prevent premature polymerization or condensation of titanates. The latter reactions are base catalyzed. The organotitanate may be mixed with an aqueous solvent system and a surfactant. The solvent/surfactant system selected should tolerate a high solid content, have good leveling or spreading capacity, and be miscible with water. Alcohols and the fluorosurfactants manufactured by 3M (Minnesota) are particularly useful for this method. Hydrolysis of the organotitanate should occur prior to any polymerization or condensation, and the solvent system should be acidic to prevent undesired polymerization reactions. The counter ion supplied by the acid can be used to improve the solubility of the titanium—acetic acid and hydrochloric acid are preferred. A nonaqueous solvent system may be used but the organotitanate can not be pre-hydrolyzed. The solvent can be anhydrous to improve the stability of the coating solution. Suitable solvents include toluene, heptane, and hexane. A surfactant can be omitted (as in the aqueous solvent system), but may further improve the coating characteristics.

Once the organotitanate and the solvent system are mixed, a predetermined volume of this solution is applied to an optical substrate using a spin coating technique. When the organotitanate is mixed with a non-aqueous solvent system, the solution is applied to the optical substrate by dynamic delivery. In a dynamic delivery method, the substrate is attached to the spin coater and spun at 4,000 to 5,000 rpm. The solution is applied to the spinning substrate which continues to spin until an even film is obtained. For aqueous solvent systems, dynamic or static delivery of the solution is possible. In static delivery, the solution is applied to the substrate and then the spinning is initiated. The spin rate required is dependent on the percent solids in the solution, the volume applied to the substrate, and the substrate size. The thickness of the titanium layer generated is a function of the percent solid, the volume applied, and the spin rate.

The titanium dioxide layer may be cured to the substrate by a number of techniques. The refractive index of the titanium dioxide layer is controlled by the temperature of the substrate during curing and to a much lesser degree the length of the curing process. The curing process may use a furnace, an infrared heat lamp, a hot plate, or a microwave oven. In addition to the titanates, silicates, aluminum alkyloxides, and the corresponding analogs of zirconium may all be used to produce an optical thin film by this method. In addition to spin coating the titanium dioxide, polysilazanes may be used to produce silicon nitride coatings by spin coating. These protocols may also be adapted for use in this technology.

Optimization Procedure

Optimizing the selection of the substrate, optical thin film, attachment layer, and receptive layer can be carried out using the procedure disclosed in U.S. Pat. No. 5,955,377, incorporated herein by reference in its entirety.

Attachment Layer

The present invention is further concerned with materials and methods for producing a layer which connects the analyte-specific binding layer to the optical substrate or optical thin film. The present invention provides a method for producing an attachment layer which optimizes the functional density, stability, and viability of receptive material immobilized on that layer.

The attachment layer is intended to provide a chemical bridge between a selected inorganic substrate material while remaining compatible with the biological or receptive materials, physically adhering or covalently attaching to the upper test surface (whether an optical thin film is included or not), preferably not interfering with the desired thin film properties of the test surface, and must being sufficiently durable to withstand subsequent processing steps.

The density and stability of immobilized receptive material (or, in some cases, enzymes) can be controlled to optimize the performance of an assay test surface.

Applicant has determined that one problem in obtaining useful devices of this invention was the extremely limited macroscopic and/or microscopic surface area of the test films employed in a thin film assay as compared with the microscopically convoluted surface characteristics of other conventional solid phase assay materials. In most cases, the optical substrate must be evenly coated with a continuous attachment layer that protects the receptive material from any toxic effects of the reflective substrate while adhering it to the surface.

In conventional solid phase assays, the larger test surfaces generally employed, such as microtiter wells, have much greater total surface area and microscopically convoluted surfaces relative to a thin film substrate. Thus, the amount of receptive material immobilized compensates for any sparsity in coverage, or any losses in viability (ability to bind analyte) which result from conformational or chemical changes caused by the immobilization process. It also compensates for any receptive material which may be unavailable for binding due to poor orientation. Thus, applicant has discovered that in direct thin film assays the surface area limitations require the use or development of special materials and procedures designed to maximize the functional density, viability, stability, and accessibility of the receptive material.

Much of the original work to adapt siliceous materials for retention of specific binding molecules originated with affinity chromatography applications and used silica (SiO2) gel, and solid supports such as glass. Initial activation of silica towards the binding material was accomplished by treatment with a dichlorodimethylsilane. Silanization, regardless of the process used to apply the silane, can introduce groups capable of covalently attaching the molecule by chemical means.

In a preferred embodiment, the attachment layer is spin coated or aerosol spray coated in a uniform manner. The various intermediate materials are coated to the substrate at thicknesses between 5 Å and 500 Å (thicker amounts can be employed). The layer can be formed of any material that performs the following functions and has the following characteristics: creates a favorable environment for the receptive material, permits the receptive material to be bound in active, functional levels (preferably by a cost-effective method), adheres tightly to the optical substrate, and can be coated uniformly.

For direct eye detection methodologies, the surface activation technique can provide a covalent modification of the surface for stability while introducing a very dense uniform or conformal film on the surface of the substrate. A strongly adsorbed conformal film without covalent attachment may be adequate for substrates, such as monocrystalline silicon, macroscopically planar, uniform optical glasses, metalized glass and plastic, whether or not coated with an optical layer (i.e., SiO, SiO2, Six Ny, etc.). Once applied, the attachment layer should provide an environment which supports the adherence of a specific binding layer by covalent or adsorptive interactions, that is dense and functional. This attachment layer must be of sufficient thickness to separate the specific binding layer from any toxic effects of the initial optical substrate.

The immobilization chemistry for attaching the receptive material to the attachment layer is selected based on the properties of both the attachment layer and the receptive material. The receptive material can be covalently or passively attached to this material. When the attachment layer is specifically adapted for covalent attachment, an additional step to activate the attachment layer may be required. A variety of activation and linking procedures can be employed. For example, photo-activated biotin can be employed to adhere the receptive material. Usually, it is sufficient to passively adsorb the receptive material to the attachment layer, thus avoiding the time and expense of immobilization chemistry procedures.

Fc-Specific Binding Layer

In accordance with the present invention, it has been shown that the use of an Fc-specific binding protein provides previously unappreciated advantages, including significantly improved sensitivity and ability to detect target analytes present in a biological sample at significantly lower concentrations. In one aspect of the present invention, there is provided a thin-film biosensor chip that includes a biologically compatible attachment layer comprising an Fc-specific binding protein that is capable of specific or selective binding to the Fc region of an immunoglobulin molecule. In other aspects of the invention, the thin-film biosensor chip of the present invention comprises an Fc-specific binding protein attached to a polypeptide layer that provides amino functional groups to the surface and facilitates attachment of other biomolecules used to adsorb proteins. Polypeptides that include amino functional groups include, for example, poly(phenylalanine-lysine). In yet another aspect of the invention, the thin-film biosensor chip of the present invention comprises an Fc-specific binding protein attached to a polypeptide layer that provides amino functional groups to the surface and facilitates attachment of other biomolecules used to adsorb proteins, and a non-polymeric silane layer.

The use of an Fc-specific binding protein provides an attachment moiety to which an antibody capture molecule specific to the target analyte of interest can bind. The use of an Fc-specific binding protein adds thickness to the attachment layer and specificity to an analyte-specific antibody used to bind and detect the target analyte of interest. In accordance with the present invention, the use of Fc-specific binding proteins are particularly advantageous in detecting target analytes that are present in biological samples in low abundance. In a particular embodiment of the invention, the thin-film biosensor chip and methods of the present invention having an attachment layer comprising an Fc-specific binding protein in combination with a non-polymeric silane layer provides additional improvements in sensitivity, enabling detection of target analytes present in a biological sample at significantly lower concentrations. Detection of low abundance analytes associated with disease will allow earlier detection of disease, as well as detection of diseases cause by infectious agents that may inherently be present in lower concentrations.

The Fc-specific binding proteins of the present invention include any proteins that are capable of binding to the Fc region of an immunoglobulin molecule. The Fc-specific binding proteins of the present invention are used as a universal antibody-binding molecule, for binding non-specifically to an antibody capture molecule specific to the target analyte of interest. Numerous Fc-specific binding proteins are known in the art. For example, Protein G from Streptococcus sp. is known to bind specifically to the Fc region of many immunoglobulins. The property of binding to the Fc region of antibodies is also seen in other bacterial proteins, such as Protein A, and Protein L. An antibody against another antibody can also be used to specifically bind an antibody capture molecule specific to the target analyte being detected. Certain complement proteins are also known to have specific antibody binding sites.

By way of example, particular Fc-specific proteins may include protein G, protein A, protein L, protein LA, C1q complement protein, Fc receptor protein, IgG3 binding protein M12, anti-Fc antibodies, and recombinant proteins that specifically bind Fc, and Fc binding fragments thereof. In one embodiment of the invention, the Fc-specific protein is protein G, a bacterial cell wall protein isolated from group G streptococci, which binds to the Fc region of most mammalian immunoglobulins, in particular gamma immunoglobulins. In another embodiment of the invention, the Fc-specific protein is protein A, a bacterial cell wall protein isolated from Staphylococcus aureus. In another embodiment of the invention, the Fc-specific protein is protein L. In another embodiment of the invention, the Fc-specific protein is protein LA. In another embodiment of the invention, the Fc-specific protein is C1q complement protein. In another embodiment of the invention, the Fc-specific protein is an Fc receptor protein. In another embodiment of the invention, the Fc-specific protein is an IgG3 binding protein M12. In another embodiment of the invention, the Fc-specific protein is an anti-Fc antibody (for example, using a goat anti-human antibody, followed by use of a human antibody against the specific target analyte to bind to the goat anti-human antibody). In another embodiment of the invention, the Fc-specific protein is a recombinant protein that specifically bindings Fc.

For purposes of use in the present invention, it is desirable to use Protein G that has been recombinantly expressed, for example, in E. coli.

A thin layer that does not change the optical activity (index of refraction) of the chip is desirable. However, the layer must also be thick enough to attach an optimal number of antibodies.

Receptive Material

Receptive materials can include one part of a specific binding pair such as antigen/antibody, enzyme/substrate, oligonucleotide/DNA, chelator/metal, enzyme/inhibitor, bacteria/receptor, virus/receptor, hormone/receptor, DNA/RNA, or RNA/RNA, oligonucleotide/RNA, and binding of these species to any other species, as well as the interaction of these species with inorganic species.

The receptive material that is bound to the attachment layer can be characterized by an ability to specifically bind the analyte or analytes of interest. There is a wide variety of materials that can be used as receptive material, which is limited only by the types of material which will combine selectively (with respect to any chosen sample) with a secondary partner. Subclasses of materials which can be included in the overall class of receptive materials includes toxins, antibodies, antigens, hormone receptors, parasites, cells, haptens, metabolites, allergens, nucleic acids, nuclear materials, autoantibodies, blood proteins, cellular debris, enzymes, tissue proteins, enzyme substrates, co-enzymes, neuron transmitters, viruses, viral particles, microorganisms, proteins, polysaccharides, chelators, drugs, and any other member of a specific binding pair. This list only incorporates some of the many different materials that can be coated onto the attachment layer to produce a thin film assay system. Whatever the selected analyte of interest is, the receptive material is designed to bind specifically with the analyte of interest.

The matrix containing the analyte of interest may be a fluid, a solid, a gas, or a bodily fluid such as mucous, saliva, urine, fecal material, tissue, marrow, cerebral spinal fluid, serum, plasma, whole blood, sputum, buffered solutions, extracted solutions, semen, vaginal secretions, pericardial, gastric, peritoneal, pleural, or other washes and the like. The analyte of interest may be an antigen, an antibody, an enzyme, a DNA fragment, an intact gene, a RNA fragment, a small molecule, a metal, a toxin, an environmental agent, a nucleic acid, a cytoplasmic component, pili or flagella component, protein, polysaccharide, drug, or any other material. For example, receptive material for bacteria may specifically bind a surface membrane component—protein or lipid, a polysaccharide, a nucleic acid, or an enzyme. The analyte which is specific to the bacteria may be a polysaccharide, an enzyme, a nucleic acid, a membrane component, or an antibody produced by the host in response to the bacteria. The presence of the analyte may indicate an infectious disease (bacterial or viral), cancer or other metabolic disorder or condition. The presence of the analyte may be an indication of food poisoning or other toxic exposure. The analyte may indicate drug abuse or may monitor levels of therapeutic agents. The analyte may also be an indication of some other condition or biological activity or property.

One of the most commonly encountered assay protocols for which this technology can be utilized is an immunoassay. The discussion presented for construction of a receptive material layer hereafter specifically addresses immunoassays. However, the general considerations apply to nucleic acid probes, enzyme/substrate, and other ligand/receptor assay formats. For immunoassays, an antibody may serve as the receptive material or it may be the analyte of interest. The receptive material, for example an antibody, can form a stable, dense, reactive layer on the attachment layer of the biosensor device. If an antigen is to be detected and an antibody is the receptive material, the antibody can be specific to the antigen of interest, and the antibody (receptive material) can bind the antigen (analyte) with sufficient avidity that the antigen is retained at the biosensor surface. In some cases, the analyte may not simply bind the receptive material, but may cause a detectable modification of the receptive material to occur. This interaction could cause an increase in mass at the biosensor surface or a decrease in the amount of receptive material on the biosensor surface. An example of the latter is the interaction of a degradative enzyme or material with a specific, immobilized substrate. The specific mechanism through which binding, hybridization, or interaction of the analyte with the receptive material occurs is not important but may impact the reaction conditions used in the final assay protocol.

In general, the receptive material may be passively adhered to the attachment layer. If required, the free functional groups introduced onto the biosensor surface by the attachment layer may be used for covalent attachment of receptive material to the biosensor surface. Chemistries available for attachment of receptive materials are well known to those skilled in the art.

A wide range of techniques can be used to adhere the receptive material to the attachment layer. Biosensor surfaces may be coated with receptive material by. For example, total immersion in a solution for a pre-determined period of time, application of solution in discrete arrays or patterns, spraying, ink jet, or other imprinting methods, or by spin coating from an appropriate solvent system. The technique selected should minimize the amount of receptive material required for coating a large number of biosensor surfaces and maintain the stability/functionality of receptive material during application. The technique can also apply or adhere the receptive material to the attachment layer in a very uniform and reproducible fashion.

Composition of the coating solution will depend on the method of application and type of receptive material to be utilized. If a spin coating technique is used, a surfactant may improve the uniformity of the receptive material across the optical substrate or support. In general, the coating solution will be a buffered aqueous solution at a pH, composition, and ionic strength that promotes passive adhesion of the receptive material to the attachment layer. The exact conditions selected will depend on the type of receptive material used for the assay under development. Once coating conditions are established for a particular type of receptive material, e.g., polyclonal antibodies, these conditions are suitable for all assays based on such receptive material. However, chemically distinct receptive materials, for example polyclonal antibodies and nucleic acids, may not coat equally well to the attachment layer under similar buffer and application conditions.

The materials and methods described above allow the construction of a specific binding biosensor surface. The biosensor surface is composed of an optical substrate or support, an optional optical thin film, an attachment layer, and finally a binding layer. For a visual determination of a specific binding event or interaction, the composite interference film can be designed to include the optical thin film, the attachment layer and the binding layer. The initial interference color selected can be maintained when the attachment layer and receptive material are coated onto the optical thin film. Once a surface is coated with the binding layer, a small spot of a preparation containing the analyte of interest may be applied to the surface. This is incubated for a few minutes, rinsed, and then dried such as by a stream of nitrogen. This will generate a procedural control which will be developed whether the sample being assayed is positive or negative. This control assures the end-user, that the assay protocol was followed correctly and that all the reagents in the kit are performing correctly. The procedural control may be applied in any pattern desired.

Like the procedural control the receptive material may be applied in a pattern. Thus, the device can provide a visual symbol in response to polychromatic light when the optical thin film is applied to the optical substrate. The coating solution containing receptive material may be applied to the surface which is covered with a mask. The mask allows the receptive material to be immobilized on the attachment layer only in the sections which are exposed to the coating solution. A surface which is uniformly coated with receptive material may be covered with a mask, and the receptive material may be selectively inactivated. There are a number of techniques which are suitable for the inactivation of receptive material. One of the simplest techniques for biological materials is to expose section of the receptive material to UV irradiation for a sufficient period of time to inactive the material. The mask may be designed in any pattern which will assist the end-user in interpretation of the results.

Techniques such as stamping, ink jet printing, ultra-sonic dispensers, and other liquid dispensing equipment are suitable for generation of a pattern of the receptive material. The receptive material may be applied in the pattern by these techniques, incubated for a period of time, and then rinsed from the surface. Exposed sections of attachment material may be coated with an inert material similar to the receptive material.

A particularly useful combination of interference colors relies on a yellow/gold interference color for the biosensor surface background or starting point. Since mass is a function of thickness and concentration, when an increase in mass occurs at the surface, the reacted zone changes interference color to a purple/blue color. As described above, the optical thin film can be adjusted and optimized to compensate for the layers required in the construction of the biological biosensor surface to maintain the desired starting interference color.

Mass Enhancement

Thin-film detection methods which provide direct determination of specific binding pairs offer significant advantages in contrast to radioactive or enzymatic means, including fluorescent, luminescent, calorimetric, or other tag-dependent detection schemes. Thin-film systems can be applied in the detection of small molecules. Such analytes, however, fail to produce sufficient thickness or optical density for direct eye or instrumented detection. Thin-film detection systems can perform optimally when the integrity of the film is maintained. Thus, a method designed for amplification in such a system can provide an increase in thickness or mass and maintain the film integrity, as well as meet a limitation imposed by the detection system, and can be of the simplest possible construction.

The amplification technique may be directly related to the concentration of the analyte of interest or may be inversely proportional to the concentration of the analyte of interest as in a competitive or inhibition assay format. The binding of a mass enhancement or amplification reagent can be a specific function of the analyte binding to the biosensor surface and may be considered as part of a signal generating reagent.

The mass enhancement reagent can be capable of passive or covalent attachment to a secondary receptive material. An example of passive attachment to a mass enhancing reagent is the adsorption of antibodies onto surface activator particles. An example of the covalent attachment of a mass enhancing reagent to the secondary receptive material is the conjugation of horseradish peroxidase (HRP, or another enzyme) to an antibody. Other enzymes are discussed in U.S. Pat. No. 5,955,377, incorporated herein by reference in its entirety, may be used. Regardless of the mechanism employed, the mass enhancement reagent should form a stable product or adduct with the secondary receptive material. The coupling protocol selected should not leave or introduce non-specific binding effects at the biosensor surface. The mass enhancement reagent may also be capable of direct, specific interaction with the analyte.

Thus, in another aspect, methods for the amplification of signals in assay systems which rely on a thin-film detection method as disclosed. Such methods include, but are not limited to, ellipsometry, interference effects, profilometry, scanning tunneling microscopy, atomic force microscopy, interferometry, light scattering, total internal reflection, or reflectometric techniques. The materials selected for use in these types of systems preferably maintain some degree of particulate character in solution, and upon contact with a surface or support form a stable thin film. The film can be conformal to the biosensor surface to maintain the desired smoothness or texture of the substrate. The characteristic texture of the surface will be dependent on the detection method employed. The material selected can also be capable of adhering, through covalent or passive interaction, a receptive material or one member of a specific binding pair. A secondary receptive material or binding reagent can be adhered to the signal amplifying material or particle in a manner which preserves the reactivity and stability of the secondary receptive material. The secondary receptive material applied to the particle may be identical to, or matched to the receptive material immobilized on the biosensor surface. The combination of a secondary receptive material or binding reagent and additional material, whether a particle, an enzyme, or etc., forms a mass enhancement or signal generating reagent.

In general, an optical assay where amplification benefits the assay include those assays where a substrate whose properties and characteristics are determined by the type of detection method used, an optional secondary optical material, an attachment layer, a layer of receptive material, and the mass enhancement reagent. A general assay protocol may include that the sample suspected of containing the analyte of interest be processed through any treatment necessary, such as extraction of a cellular antigen, and then be mixed with the secondary or amplification reagent. An aliquot of this mixture can be applied to the receptive material coated substrate. After an appropriate incubation period, the unbound material is separated from the reacted film by either a physical rinse/dry protocol or with a device contained rinse/dry step. The signal can then be interpreted visually or instrumentally. The introduction of the secondary or amplification reagent can be achieved by addition of a reagent to the sample as a lyophilized material in the sample collection or application device, or embedded in an assay device. Examples of precipitating enzymes include horseradish peroxidase, alkaline phosphatase, and glucose oxidase.

Catalytic Production of Solid

Enhanced sensitivity of optical thin film assays can be obtained with an enzyme/substrate pair which produces insoluble precipitated products on the thin film surface. The catalytic nature of this amplification technique improves the sensitivity of the method. Enzymes which may be useful include glucose oxidase, galactosidase peroxidase, alkaline phosphatase and the like. However, any process which provides a specific component which can be attached to a receptive material and can catalyze conversion of a substrate to a precipitated film product may be suitable. An insoluble reaction product results when immobilized antibody-antigen-antibody-HRP complex is present on the biosensor surface. A enzyme catalyzed reaction product is precipitated by the action of a precipitating agent such as combination of alginic acid, dextran sulfate, methyl vinyl ether/maleic anhydride copolymer, or carrageenan and the like, and with the product formed by the interaction of TMB (3,3′,5,5′-tetra-methyl-benzidine) with an oxygen free radical. This particular substrate will form an insoluble product whenever a free radical contacts the TMB. Other substances such as chloro-napthol, diaminobenzidene tetrahydrochloride, aminoethyl-carbazole, orthophenylenediamine and the like can also be used. These are used in concentrations from about 10 to about 100 mM. As a result, a measurable increase in mass occurs with the enzyme-conjugate layer. A variety of enzyme substrate systems or catalytic systems may be employed that will increase the mass deposited on the surface.

Referring again to FIGS. 3A-F, a graphic representation of a cross-section of the multilayer device having a substrate is shown. The upper surface of the device has various coated layers. In one example, these layers include a layer of silicon nitride immediately adjacent to the upper optical substrate layer, an attachment layer such as a nonpolymeric silane, and the receptive material, which for a bacterial antigen assay is an antibody.

If desired, the analyte of interest may be combined with the mass enhancing reagent and the immobilized receptive material either in a simultaneous or sequential addition process. Either mechanism results in the formation of an analyte/mass enhancement reagent complex which is immobilized on the biosensor surface. Thus, the mass enhancement reagent may be mixed directly with the sample. This mixture may then be applied to the reactive biosensor surface and incubated for the required period. This is a simultaneous assay format.

In some cases additional sensitivity is gained by performing a sequential addition of the sample followed by the mass enhancement reagent. Any mechanism or specific interaction can be exploited for the generation of a mass enhancement reagent. For instance, nucleic acids are known to tightly bind or intercalate a number of materials, such as metals, and certain dyes. These materials would serve to introduce mass into a specifically immobilized nucleic acid.

The increase of the product layer may be determined both visually or instrumentally, such as by ellipsometry and where light intensity differentials are caused by the increased thickness. The receptive material enzyme complex is thus capable of direct interaction with an analyte of interest and more particularly is evidence of an analyte, such as an antigen. This change is detectable by measuring the optical thickness and does not necessarily depend on any light reflectivity of the substrate material. One such instrument is the Sagax Ellipsometer, described in U.S. Pat. Nos. 4,332,476, 4,655,595, 4,647,207, and 4,558,012, which disclosures are incorporated by reference in their entirety.

Devices

Several configurations of the above multilayer biosensor surface in a device format are possible. In one embodiment, an assay format includes a single use, single sample device. In another embodiment, an assay format provides for a single sample to be screened for the presence of multiple analytes. In yet another embodiment, multiple samples can be screened for a single analyte or batch testing.

In a single use device, the device can used to test for a wide range disease state or conditions, such as infectious disease testing, pregnancy or fertility testing, etc. Protocols for using these single test devices can be very simple. The sealed device is opened, exposing the reactive biosensor surface. A sample is applied to the biosensor surface and incubated for a short period of time, for example, 2 minutes. The sample may or may not require pre-treatment, such as antigen extraction from bacteria, etc. Addition of a secondary reagent to the sample prior to application to the biosensor surface may also be required. Once the incubation period is complete, the unreacted sample is removed with a water rinse. The device is blotted to dry the biosensor surface. Depending on the biosensor and the mass enhancement/amplification method used, the assay is complete or the assay may require additional incubation/wash/dry cycles. The biosensor device and protocol can be well suited to physician office, clinical laboratory, home or field testing environments. A protective shell can also be provided around the device, e.g., composed of polystyrene, polypropylene, polyethylene, or the like, which is readily formed into a molded or injection molded devices. Multi-analyte or multi-sample devices may be made of similar materials using similar processes.

Examples of additional devices that can be used are those disclosed in U.S. Pat. No. 5,955,377 incorporated herein by reference in its entirety.

Instrumentation

After the sample is contacted with the surface of a test device, an instrument can be used to detect analyte binding. One such instrument is the Sagax Ellipsometer (see, U.S. Pat. Nos. 4,332,476, 4,655,595, 4,647,207 and 4,558,012). Alternate instruments capable of use include traditional null ellipsometers, thin film analyzers, profilometers, polarimeter, etc. If an interference film is included in the biosensor surface construction, then a simple reflectometer an be used for quantitation. Other suitable instruments are disclosed in U.S. Pat. No. 5,955,377. Instruments using plasma resonance may also be used to carry out analyte binding detection.

Analytes

A variety of analytes may be investigated. These analytes include proteins, peptides, nucleic acids, carbohydrates, glyoproteins, chelates, metal chelates, metal ligands, biotin-avidin-analyte complexes, and the like.

### EXAMPLES

### Example 1

### Attachment of Protein G to Chips

A thin-film biosensor chip was prepared having an attachment layer comprising a non-polymeric silane in combination with Protein G. Protein G was attached to silanized chips at various concentrations, ranging from 20 pg/100 μL per chip up to 800 pg/100 μL per chip, using the following protocol.

Silanized centrifuge tubes (coated with Sigma #85126 silanization solution I) were used for dilutions to prevent protein binding to the plastic tubes. Protein G (Sigma Chemical Company, Catalog #P4689-1 mg, lyophilized from a Tris-Hcl buffer) was diluted in a Phosphate Buffer Solution (PBS) of 20 mM NaPhosphate and 0.15M NaCl pH=7.2, to the following concentrations:

a. 10 μL of 1 mg/mL Protein-G+990 μL PBS=1 mL of 10 μg/mL Protein-G.

b. 10 μL of 10 μg/ml Protein-G+990 μL PBS=1 mL of 100 ng/mL Protein-G.

c. 16 μL of 100 ng/mL Protein-G+984 μL PBS=1 mL of 1.6 ng/mL Protein-G.

For applying 160 pg/100 ul/6 mm² chip, a 2% DMA-T-Silane chip was immersed in 100 μL of 1.6 ng/mL Protein-G for 1 hour, and then washed four times with PBS.

After binding of the Protein G to the silane coated chip, the capture antibody was bound to the Protein G by specific adsorption. Specifically, a capture antibody (up to ug/ml) was placed in phosphate buffered saline (20 mM Na Phos, 150 mM NaCl, 0.02% Tween 20, ph7.5) and soaked. Adsorption of antibody binds over a wide range of conditions. The Tween 20 is a surfactant that is used as wetting agent to get the protein in contact with the silane surface.

Spots of the capture antibody solution (using 100-400 nl spots of the solution) were placed on the chip and allowed to bind for several hours. Unbound antibody was then removed by aspiration with PBS (2 or more times) and rinsing with deionized water.

Unbound binding sites on the Protein Chip were then blocked with bovine antibodies found in milk by soaking the entire chip in 100 ul of 2% nonfat dry milk, 0.5% alkaline treated casein (Biostar), 0.02% Tween 20 for 1 hr. The chip was then washed with PBS (2 times or more) and rinsed with water.

Antigen mixtures were then added to the chip in 0.5% alkaline treated casein (Biostar) and 0.02% Tween 20 for 1-2 hrs. The chip was washed with PBS (two or more time) and rinsed with water and then developed with an enzyme conjugated antibody or perhaps an HRP conjugated strepavidin depending on the label. Other concentrations of Protein-G were applied to chips using substantially the same procedure.

Results showed that a rabbit anti-goat capture antibody bound to Protein G can detect 1-10 ng/ml of an HRP conjugated goat antihuman antibody. Only high concentrations of IgG developed into visible spots. Additional reactions used 80 pg/100 μL per chip and showed detectable attachment down to 2×10-6 mg/nL IgG on T-Silane chips. Testing between this concentration and others up to 800 pg/100 μL per chip suggests that 0.160 ng up to 5 ng/100 μL per 6 mm2 chip enables detection of a target analyte.

### Example 2

### Sensitivity of Chips

A thin-film biosensor chip was prepared having an attachment layer comprising a non-polymeric silane. A solution of 2% (3-aminopropyl)triethoxysilane in hexane was prepared using 200 uL of (3-aminopropyl)triethoxysilane and 10 mL. A substrate coated with silicon nitride (Si3N4) chip was submerged in the 2% (3-aminopropyl) triethoxysilane solution for 3 hours. Following submersion and incubation, the chip was washed with hexane and washed with water three times. After rinsing, the chip was air dried.

Thin-film biosensor chips were prepared according example 1. Following preparation, the chips was further modified by incubating the chip in a 100 μL of a solution containing 8 ng/mL of Protein G at room temperature for 2 hours. Following incubation, the solution was aspirated and the chips were washed twice with PBS and twice with water. The resulting chip with Protein G was incubated in 100 μL of a solution containing 5 μg/mL of Goat anti-TNFα antibody at room temperature for 17 hours. Following incubation with the antibody, the solution was aspirated and the resulting chips were washed twice with PBS and twice with water. The resulting chip was next contacted with a solution containing TNFα protein antigen, mouse anti-TNFα monoclonal antibody with 2% non-fat dry milk, 0.5% ATC, and 0.02% Tween 20.

A) 205 pg/200 nl and 500 pg of the monoclonal

B) 41 pg/200 nl and 500 pg of the monoclonal

C) 8.2 pg/200 nl and 500 pg of the monoclonal

D) 1.6 pg/200 nl and 500 pg of the monoclonal

E) 330 fg/200 nl and 500 pg of the monoclonal

X) Control marker, mouse conjugated HRP antibody 1 ug/ml.

Next, the chips were spotted with 200 nanoliters of each solution mixture and incubated for 5 hours. Following spot arrangement, the monoclonal antibody-antigen mixtures were aspirated and washed twice with PBS, and twice with water. Following washing, the chips were incubated in 100 μL of 2% non-fat dry milk, 0.5% ATC, and 0.02% Tween 20 for 10 minutes. The solution was then aspirated and washed twice with PBS, and twice with water. The resulting chips were then incubated in 100 μL of 1 μg/ml goat anti-mouse antibody with a conjugated HRP (diluted in 2% non-fat dry milk, 0.5% ATC, and 0.02% Tween 20 for 1 hour. Following incubation, the solution was aspirated and washed twice with PBS and twice with water. Next, the chips were incubated with 100 μL of small particle TMB for 10 minutes. Following incubation, the solution was aspirated and the chips were washed 4 times with water. Following washing, the chips were visually inspected chip. A color change was observed for spots A, B, C, D but not E and X. Positive indication for the presence of the screened analyte was detected to the 1.6 picogram level (D spot).

In some embodiments, an incubation can be performed with no alkaline treated casein, in 5% non-fat dry milk and 0.01% Tween prior to aspiration and washing.

1. A thin-film biosensor chip for detecting a target analyte in a biological sample, comprising:
a solid substrate;
an antireflective optical layer coating the substrate;
an attachment layer comprising a non-polymeric silane non-covalently coupled to the antireflective optical layer.

2. The thin-film biosensor chip according to claim 1, further comprising:
an amino-functional polypeptide layer coupled to the attachment layer.

3. (canceled)

4. The thin-film biosensor chip according to claim 1, further comprising an Fc-specific binding molecule.

5-6. (canceled)

7. The thin-film biosensor chip according to claim 4, wherein the Fc-specific binding molecule is selected from the group consisting of: protein G, protein A, protein L, protein LA, C1q complement protein, Fc receptor protein, IgG3 binding protein M12, anti-Fc antibodies, and recombinant proteins that specifically bind Fc.

8. The thin-film biosensor chip according to claim 7, wherein the Fc-specific binding molecule is protein G.

9. The thin-film biosensor chip according to claim 1, further comprising a first binding molecule, wherein the first binding molecule can bind the target analyte.

10-19. (canceled)

20. The thin-film biosensor chip according to claim 9, wherein the first binding molecule is an antibody.

21. The thin-film biosensor chip according to claim 1, further comprising a reflective layer coating the substrate and underlying the antireflective optical layer.

22-30. (canceled)

31. A kit for a thin-film biosensor assay for detecting a target analyte in a biological sample, comprising:
a thin-film biosensor chip comprising:
(a) a solid substrate;
(b) an antireflective optical layer coating the substrate;
(c) an attachment layer comprising a non-polymeric silane non-covalently coupled to the antireflective optical layer.

32. The kit according to claim 31, wherein the thin-film biosensor chip further comprises an amino-functional polypeptide layer coupled to the attachment layer.

33. (canceled)

34. The kit according to claim 31, wherein the thin-film biosensor chip further comprises an Fc-specific binding molecule coupled to the non-polymeric silane.

35-36. (canceled)

37. The kit according to claim 34, wherein the Fc-specific binding molecule is selected from the group consisting of: protein G, protein A, protein L, protein LA, C1q complement protein, Fc receptor protein, IgG3 binding protein M12, anti-Fc antibodies, and recombinant proteins that specifically bind Fc.

38. The kit according to claim 37, wherein the Fc-specific binding molecule is protein G.

39. The kit according to claim 31, further comprising a first analyte-specific binding molecule capable of binding the target analyte.

40-48. (canceled)

49. The kit according to claim 39, wherein the first analyte-specific binding molecule is an antibody.

50-51. (canceled)

52. The kit according to claim 39, wherein the thin-film biosensor chip further comprises a reflective layer coating the substrate and underlying the antireflective optical layer.

53-62. (canceled)

63. A method of preparing a thin-film biosensor chip for detecting a target analyte in a biological sample, comprising:
providing a solid substrate;
coating the substrate with an antireflective optical layer;
contacting the antireflective optical layer with a non-polymeric silane to non-covalently couple the antireflective optical layer with a non-polymeric silane.

64. The method according to claim 63, further comprising contacting the non-polymeric silane with an amino-functional polypeptide layer.

65. (canceled)

66. The method according to claim 63, further comprising contacting the non-polymeric silane with an Fc-specific binding molecule.

67-68. (canceled)

69. The method according to claim 66, wherein the Fc-specific binding molecule is selected from the group consisting of: protein G, protein A, protein L, protein LA, C1q complement protein, Fc receptor protein, IgG3 binding protein M12, anti-Fc antibodies, and recombinant proteins that specifically bind Fc.

70. The method according to claim 69, wherein the Fc-specific binding molecule is protein G.

71. The method according to claim 63, further comprising contacting the non-polymeric silane with a first analyte-specific binding molecule capable of binding the target analyte.

72-78. (canceled)

79. The method according to claim 63, wherein the first analyte-specific binding molecule is an antibody.

80. (canceled)

81. The method according to claim 63, further comprising coating the substrate with a reflective layer underlying the antireflective optical layer.

82-93. (canceled)

94. An optical assay method for detecting a target analyte in a biological sample, comprising:
providing a thin-film biosensor chip comprising:
a substrate;
an antireflective optical layer coating the substrate;
an attachment layer comprising a non-polymeric silane non-covalently coupled to the optical layer;
contacting the chip with the sample.

95. The method according to claim 94, wherein the thin-film biosensor chip further comprises an amino-functional polypeptide layer coupled to the attachment layer.

96. (canceled)

97. The method according to claim 94, wherein the thin-film biosensor chip further comprises an Fc-specific binding molecule coupled to the non-polymeric silane.

98-99. (canceled)

100. The method according to claim 97, wherein the Fc-specific binding molecule is selected from the group consisting of: protein G, protein A, protein L, protein LA, C1q complement protein, Fc receptor protein, IgG3 binding protein M12, anti-Fc antibodies, and recombinant proteins that specifically bind Fc.

101. The method according to claim 100, wherein the Fc-specific binding molecule is protein G.

102. The method according to claim 94, further comprising providing a first analyte-specific binding molecule capable of binding the target analyte.

103-110. (canceled)

111. The method according to claim 94, wherein the first analyte-specific binding molecule is an antibody.

112-113. (canceled)

114. The method according to claim 94, wherein the thin-film biosensor chip further comprises a reflective layer coating the substrate and underlying the antireflective optical layer.

115-125. (canceled)
